(19) United States
(12) Patent Application Publication
(10) Pub. No.: US 2006/0147999 A1
(43) Pub. Date: Jul. 6, 2006

(54) Method and apparatus for homology-based complex detection in a protein-protein interaction network

(76) Inventors: Jae Hun Choi, Daejeon (KR); Jong Min Park, Jeonju (KR); Jae Young Jung, Daejeon (KR); Seon Hee Park, Daejeon (KR)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/296,886

(22) Filed: Dec. 7, 2005

(30) Foreign Application Priority Data
Dec. 8, 2004 (KR) 2004-102915
Dec. 22, 2004 (KR) 2004-110350

(51) Int. Cl. G01N 33/53; G06F 19/00

(52) U.S. Cl. 435/7.1; 702/19

(57) ABSTRACT

Provided are a method and a apparatus for detecting a protein complex using a similarity between different proteins in a protein-protein interaction network. The method includes: (a) producing a virtual complex of a specific organism by mapping proteins contained in a complex of a different organism into proteins of the specific organism using homology information between different proteins; and (b) searching for the produced virtual complex in the protein-protein interaction network of the specific organism.

### CROSS-REFERENCE TO RELATED APPLICATION

This application claims priority to and the benefit of Korean Patent Application Nos. 2004-102915, filed Dec. 8, 2004, and 2004-110350, filed Dec. 22, 2004, the disclosures of which are incorporated herein by reference in their entirety.

### BACKGROUND

1. Field of the Invention

The present invention relates to a protein-protein interaction network in the field of bioinformatics, and more particularly, to a method and apparatus for homology-based complex detection in a protein-protein interaction network.

2. Discussion of Related Art

Generally, a protein-protein interaction (PPI) network is used as important information in the investigation of biological mechanisms. A function of a specific protein that is not identified in a PPI network can be inferred from another protein that interacts with the specific protein. Also, influence on a living body can be predicted by suppressing or activating a function of the protein.

A complex means a protein complex, and proteins contained in the complex are in charge of a complex function of a living body while interacting closely with each other in a cell. There are many complexes in a PPI network, and a complex is discovered through various biological experiments such as “co-immunoprecipitation” or “purification by molecular weight”.

Research into a method detecting a complex in a PPI network is classified into two types. The first type employs a method for searching for protein complexes through biological experimentations in a lower organism. Currently, network data and complex data obtained from the biological experiments have been well organized. However, the biological experiments are costly, and therefore a technique using homology relationships with previously discovered complexes is required.

The second type of research is for predicting and building a PPI network of a specific living body from a genome sequence, expression, or interaction data of different living bodies that have been previously discovered using information technology (IT). However, this does not include research for discovering a complex in a vast PPI network of a higher organism using IT. That is, a costly biological experiment, which has been performed for a lower organism, should be performed once more to discover a protein complex of a higher organism. Thus, there is a need for a method for detecting a complex that exists in a PPI network of a higher organism using already-discovered complex data of a lower organism.

### SUMMARY OF THE INVENTION

The present invention is directed to a method and an apparatus for detecting a complex in a PPI network of a specific organism using protein complex data already discovered in a different organism and different protein homology data.

One aspect of the present invention provides a method for detecting a complex in a PPI network, comprising: (a) producing a virtual complex of a specific organism by mapping a protein contained in a complex of a different organism into a protein of the specific organism using homology information between two proteins; and (b) searching for the virtual complex in a PPI network of the specific organism.

Step (a) may comprise: (a1) mapping proteins that make up the complex of the different organism into homology proteins of the specific organism; (a2) mapping interaction relations between the proteins that make up the complex of the different organism into interaction relations between the homology proteins of the specific organism; and (a3) producing the virtual complex using the mapped homology proteins and the mapped interaction relations between the homology proteins.

Step (b) may comprise: (b1) mapping homology proteins that make up the virtual complex into proteins contained in the PPI network; (b2) producing proteins that are not mapped in the PPI network; (b3) mapping relations between proteins that make up the virtual complex to relations between proteins contained in the PPI network; (b4) producing in the PPI network relations between proteins that are not mapped; and (b5) searching for a candidate complex corresponding to the virtual complex in the PPI network using the mapped proteins and the mapped relations between the proteins.

Steps (b2) and (b4) may further comprise providing a user with information for producing the proteins that are not mapped and the relations between the proteins that are not mapped.

Another aspect of the present invention provides a apparatus for searching for a complex in a PPI network, comprising: producing means for producing a virtual complex of a specific organism by mapping proteins contained in a complex of a different organism to proteins of the specific organism using homology information between different proteins; and searching means for searching for the virtual complex in a PPI network of the specific organism.

Preferably, the producing means maps proteins that make up the complex of a different organism into homology proteins of the specific organism, maps interaction relations between the proteins that make up the complex of a different organism into interaction relations between the homology proteins of the specific organism, and produces the virtual complex using the mapped homology proteins and the mapped interaction relations between the homology proteins.

Preferably, the searching means maps homology proteins that make up the virtual complex to proteins contained in the PPI network, produces proteins that are not mapped in the PPI network, maps relations between proteins that make up the virtual complex to relations between proteins contained in the PPI network, produces relations between proteins that are not mapped in the PPI network, and searches for a candidate complex corresponding to the virtual complex in the PPI network using the mapped proteins and the mapped relations between the proteins.

The apparatus may further comprise an input/output (I/O) means for providing a user with information for producing the proteins that are not mapped and the relations between the proteins that are not mapped. BRIEF DESCRIPTION OF THE DRAWINGS

The above and other features and advantages of the present invention will become more apparent to those of ordinary skill in the art by describing in detail exemplary embodiments thereof with reference to the attached drawings in which:

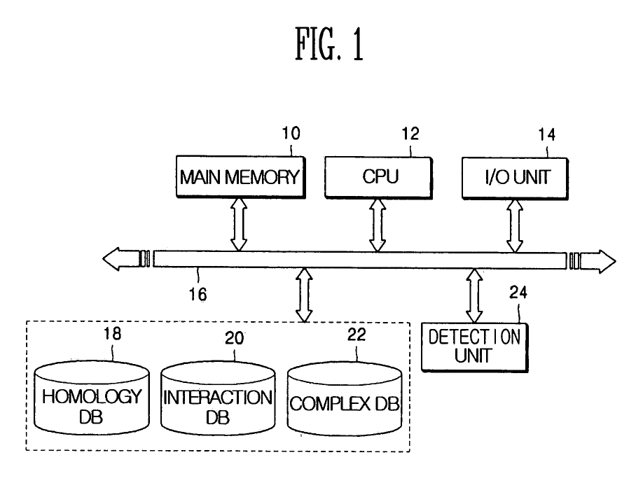

FIG. 1 is a schematic diagram of a hardware system for detecting a complex in a PPI network according to an exemplary embodiment of the present invention;

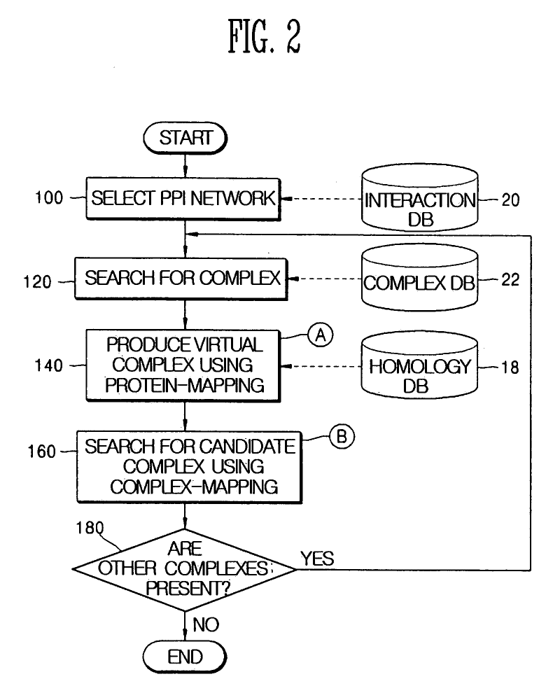

FIG. 2 is a flowchart illustrating a method for detecting a complex according to an exemplary embodiment of the present invention;

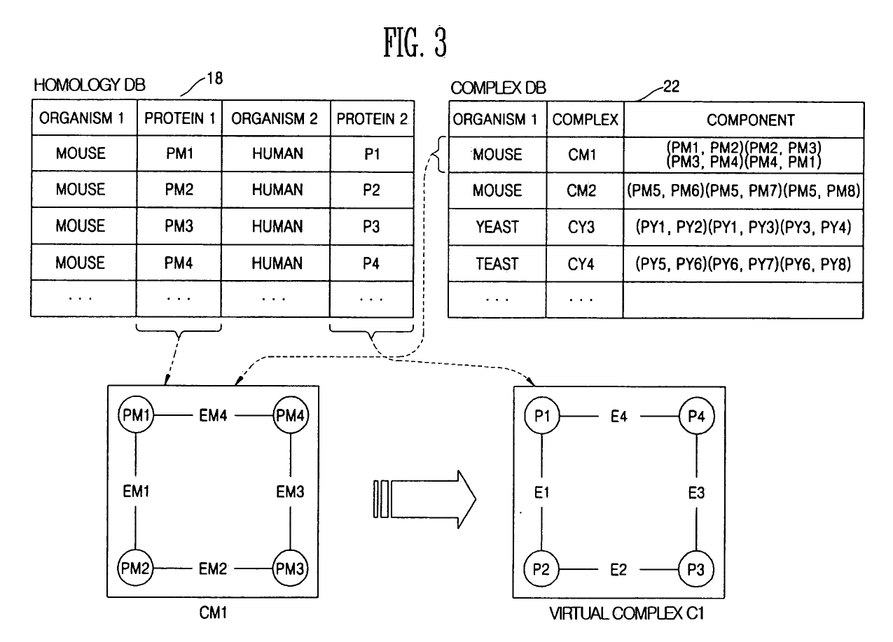

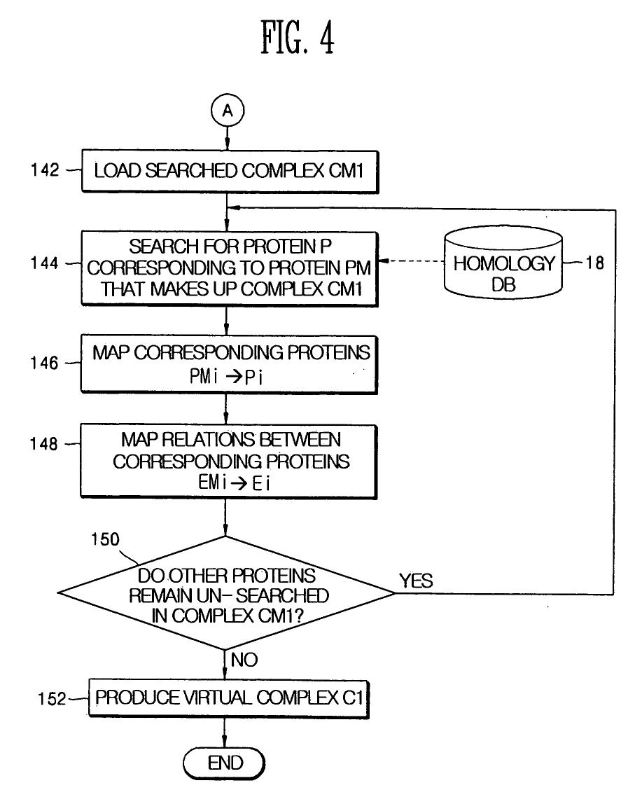

FIGS. 3 and 4 are diagrams illustrating an example and detailed procedure A for producing a virtual complex using protein mapping of FIG. 2; and

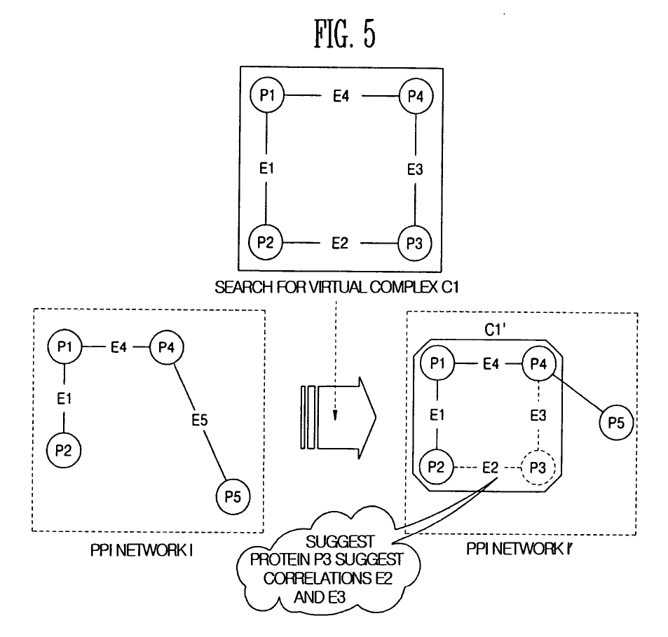

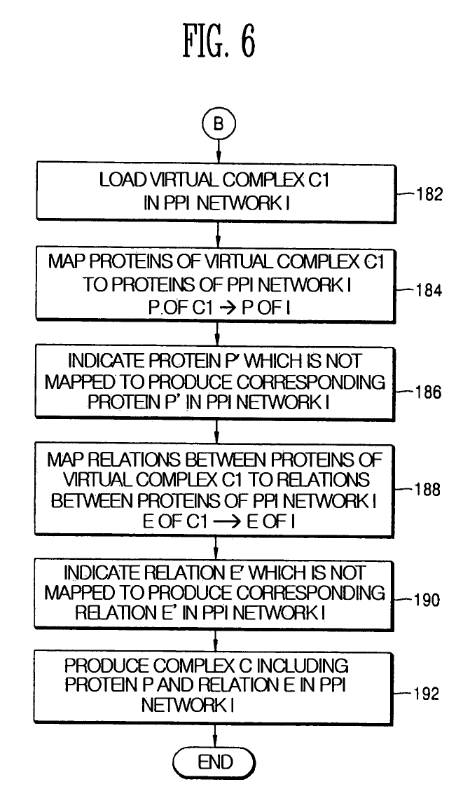

FIGS. 5 and 6 are diagrams illustrating an example and detailed procedure B for searching for detecting a candidate complex using complex mapping of FIG. 2.

### DETAILED DESCRIPTION OF EXEMPLARY EMBODIMENTS

Hereinafter, exemplary embodiments of the present invention will be described in detail. However, the present invention is not limited to the exemplary embodiments disclosed below, but can be implemented in various types. The present exemplary embodiments are provided for complete disclosure of the present invention and to fully inform the scope of the present invention to those ordinarily skilled in the art.

FIG. 1 is a schematic diagram of a hardware system for detecting a complex in a PPI network according to an exemplary embodiment of the present invention.

Referring to FIG. 1, the hardware system for detecting a complex in a PPI network according to the present invention comprises a main memory 10, a central processing unit 12, an I/O unit 14, a homology database 18, an interaction database 20, a complex database 22, a complex detection unit 24, and a system bus 16.

The main memory 10 stores complex detection system and information of the homology database 18, the interaction database 20, and the complex database 22 which are used in each step for detecting a complex. The central processing unit 12 processes the complex detection system information stored in the main memory 10 in each step, and the I/O unit 14 receives information required in the system from a user and outputs information about a complex detected by the system on a screen. Here, messages or information are transmitted between the components via the system bus 16. The complex detection unit 24 searches for a complex in a PPI network of a specific organism using protein complex data already discovered in a different organism and different protein homology data.

In particular, the homology database 18 stores information for mapping proteins contained in a selected complex to corresponding homology proteins of a different organism in the PPI network. That is, the homology database 18 stores information representing a similarity relation between a protein of a specific organism and a protein of a corresponding other organism. The interaction database 20 stores information about the PPI network, and KEGG or INTERACT can be used as the interaction database 20. The complex database 22 contains a complex that exists in a specific organism and a list of pairs of two proteins that make up the complex. A structure of each database will be explained in detail later.

A method for detecting a complex in a PPI network using the above-described hardware configuration will be explained below in detail.

FIG. 2 is a flowchart illustrating a method for detecting a complex according to the present invention.

Referring to FIG. 2, in order to detect a complex in a PPI network, a specific PPI network is selected from the interaction database 20 (step 100). At this time, the specific PPI network to be searched for can be input from a user through the I/O unit 14. The complex database 22 is searched to select a complex that can belong to the PPI network that is selected or input in step 100 (step 120). Different proteins contained in the complex selected from the homology database 18 are mapped into the homology proteins of the same organism as the PPI network, and correlation thereof is adjusted to produce a virtual complex (step 140). The interaction database 20 is searched to see whether or not the produced virtual complex exists in the PPI network (step 160). Whether or not the proteins that make up the virtual complex exist in the PPI network and whether there are relations between the proteins in the PPI network are determined. If the proteins are not part of the PPI network, proteins and relations between the proteins that are necessary for making up the virtual complex are indicated to a user, proteins and relations between the proteins that are necessary for the PPI network are produced, and a real complex (also called a candidate complex) is made up in the PPI network and displayed on a screen. As long as a complex to be searched for still exists in the PPI network, steps 120 to 180 are repeated.

FIGS. 3 and 4 are diagrams illustrating an example and detailed procedure A for producing a virtual complex using the protein mapping of FIG. 2.

FIG. 3 shows an example illustrating a detailed procedure A for producing the virtual complex of step 140 of FIG. 2. As shown in FIG. 3, the homology database 18 stores information about a corresponding relation, i.e., a homology relation between a protein PROTEIN1 contained in a specific organism ORGANISM1 and a protein PROTEIN2 of another similar organism ORGANISM2. The complex database 22 stores information about complexes in the specific organism and components which make up the complexes. A complex includes pairs of two proteins which exist in a corresponding organism.

A procedure for searching for a complex similar to a complex CM1 belonging to a mouse in a PPI network of a human organism will be explained as an example. In order to search for a complex similar to a complex CM1 of a mouse in a PPI network of a human, all proteins contained in the complex CM1 are mapped into human proteins using the homology database 18. Relations between the mouse proteins contained in the complex CM1 are mapped into relations between the human proteins. A virtual complex C1 is produced by using the mapped proteins and relations between the mapped proteins.

Referring to FIG. 3, it can be understood that the complex CM1 of the mouse comprises four protein pairs (PM1,PM2), (PM2,PM3), (PM3,PM4), and (PM4,PM1) which are stored in the complex database 22. All proteins contained in the complex CM1 are mapped into proteins of the human using the homology database 18. For example, the protein PM1 contained in the complex CM 1 of the mouse is mapped into the protein PI of the human with reference to the homology database 18 since it relates to the protein PI of the human. In the same way, the proteins PM2, PM3, and PM4 of the mouse are respectively mapped into the proteins P2, P3, and P4 of the human.

As shown in the complex database 22, a relation EMI between the proteins PM1 and PM2 of the mouse is mapped into a relation El of the proteins P1 and P of the human. In the same way, relations EM2, EM3, and EM4 between proteins of the mouse are respectively mapped into relations E2, E3, and E4 between proteins of the human. The virtual complex C1 produced as the mapping result is shown on a lower right side of FIG. 3. It can be conjectured that a complex similar to the virtual complex C1 may exist in the PPI network of the human since there is a high probability that a protein belonging to a specific organism exists in the human.

FIG. 4 is a detailed flowchart illustrating the procedure A for producing the virtual complex using the protein mapping of FIG. 2. The complex CM1 searched for in step 120 of FIG. 2 is loaded (step 142). A protein P of a corresponding organism corresponding to a protein PM which makes up the complex CM1 is retrieved from the homology database 18 (step 144). All proteins PMi are mapped into proteins Pi of the corresponding organism (step 146). Relations EMi related to the protein PM are mapped into relations Ei related to the protein P of the corresponding organism (step 148). The above-described procedure is repeated for all proteins that make up the complex CM1 (step 150), thereby finally producing the virtual complex C1 (step 152).

FIGS. 5 and 6 are diagrams illustrating an example and detailed procedure B for searching for a candidate complex using the complex mapping of FIG. 2.

FIG. 5 shows an example of the candidate complex searching procedure B shown in step 160 of FIG. 2. It is assumed that the virtual complex C1 is searched for in a PPI network I. First, all proteins Pi which make up the virtual complex C1 are mapped into proteins Pi which exist in the PPI network I. At this time, the proteins that are not mapped are indicated to a user to provide information for making up a complete complex. For example, the protein P1 contained in the virtual complex C1 is mapped into the same protein P1 in the PPI network I, but the protein P4 contained in the virtual complex C1 is not mapped into any protein in the PPI network I, and so information that the protein P4 is necessary for the PPI network I is indicated to the user to produce the same complex as the virtual complex C1 in the PPI network I.

In the same way, relations Ei between all proteins contained in the virtual complex C1 are mapped into relations Ei in the PPI network I. Information about relations which are not mapped is indicated to the user, so that the virtual complex C1 is mapped into the PPI network I by setting a new relation. For example, a relation El of the virtual complex C1 is mapped into the same relation E1 in the PPI network I, but a relation E4 of the virtual complex C1 is not mapped into the PPI network I. So, information that the relation E4 is necessary for the PPI network I is indicated to the user, so that the relation E4 is produced in the PPI network I, thereby mapping the virtual complex C1 into the PPI network I.

FIG. 6 is a detailed flowchart illustrating the candidate complex searching procedure B using the complex mapping of FIG. 2. The complex CM1 produced in step 140 of FIG. 2 is loaded in the PPI network I (step 182), and all proteins of the virtual complex C1 are respectively mapped into proteins of the PPI network I (step 184). That is, proteins Pi which make up the virtual complex C1 are mapped into proteins Pi of the PPI network I. If a protein P′ is not mapped in the above-described protein mapping procedure, non-mapped protein P′ information is indicated to a user to produce the corresponding protein P′ in the PPI network I, thereby mapping all proteins that make up the virtual complex C1 to the PPI network I.

A relation Ei between proteins of the virtual complex C1 is mapped into a relation Ei between proteins of the PPI network I (step 188). If there is a relation E′ that is not mapped, non-mapped relation E′ information is indicated to the user to produce the corresponding relation E′ in the PPI network I (step 190), thereby mapping all relations between all proteins that make up the virtual complex C1 to the PPI network I. Finally, candidate complexes (real complexes) are produced using the proteins Pi and the relations Ei between the proteins Pi which are mapped into the PPI network (step 192).

Through the above-described procedures, it is possible to search or make up a candidate complex in the PPI network of a corresponding organism using different complex data and protein homology data. Also, information about absent proteins or correlations between proteins can be indicated to a user to complete the complex.

The method for detecting a complex in the PPI network according to the present invention can be implemented by a computer program. Codes and code segments making up the computer program can be inferred easily by a computer programmer with knowledge in the field of the present invention. The computer program can be stored in a computer-readable medium and read and executed by a computer to implement the method for detecting a complex in the PPI network. The computer-readable medium includes magnetic recording media, optical recording media, and carrier waves.

As described above, the present invention provides a method for detecting a complex in the PPI network of a specific organism using protein complex data already discovered in a different organism and different protein homology data.

Thus, it is possible to automatically detect a complex of a specific higher organism using genome information of a lower organism that has already been discovered, without costly biological experiments. The complex detection method of the present invention can be effectively used in high value-added research such as new medicine development.

While the invention has been shown and described with reference to certain exemplary embodiments thereof, it will be understood by those skilled in the art that various changes in form and details may be made therein without departing from the spirit and scope of the invention as defined by the appended claims.

What is claimed is:

1. A method for detecting a complex in a protein-protein interaction (PPI) network, comprising:
(a) producing a virtual complex of a specific organism by mapping proteins contained in a complex of a different organism into proteins of the specific organism using homology information between different proteins; and
(b) searching for the produced virtual complex in the PPI network of the specific organism.

2. The method of claim 1, wherein step (a) comprises:
(a1) mapping proteins that make up the complex of the different organism into homology proteins of the specific organism;
(a2) mapping interaction relations between the proteins that make up the complex of the different organism into interaction relations between the homology proteins of the specific organism; and
(a3) producing the virtual complex using the mapped homology proteins and the mapped interaction relations between the homology proteins.

3. The method of claim 2, wherein step (b) comprises:
(b1) mapping homology proteins that make up the virtual complex into proteins contained in the PPI network;
(b2) producing portions of proteins that are not mapped in the PPI network;
(b3) mapping relations between proteins that make up the virtual complex to relations between proteins contained in the PPI network;
(b4) producing relations between the proteins that are not mapped in the PPI network; and
(b5) searching for a candidate complex corresponding to the virtual complex in the PPI network using the mapped proteins and the relations between the proteins.

4. The method of claim 3, wherein the steps (b2) and (b4) further comprise providing a user with information for producing the proteins that are not mapped and the relations between the proteins that are not mapped.

5. An apparatus for detecting a complex in a PPI network, comprising:
producing means for producing a virtual complex of a specific organism by mapping proteins contained in a complex of a different organism into proteins of the specific organism using homology information between different proteins; and
searching means searching for the produced virtual complex in the PPI network of the specific organism.

6. The apparatus of claim 5, wherein the producing means maps the proteins that make up the complex of the different organism into homology proteins of the specific organism, maps interaction relations between the proteins that make up the complex of the different organism to interaction relations between the homology proteins of the specific organism, and produces the virtual complex using the mapped homology proteins and the mapped interaction relations between the homology proteins.

7. The apparatus of claim 6, wherein the searching means maps homology proteins that make up the virtual complex into proteins contained in the PPI network, produces portions of proteins that are not mapped in the PPI network, maps relations between the proteins that make up the virtual complex to relations between the proteins contained in the PPI network, produces relations between the proteins that are not mapped in the PPI network, and searches for a candidate complex corresponding to the virtual complex in the PPI network using the mapped proteins and the mapped relations between the proteins.

8. The apparatus of claim 7, further comprising an I/O means for providing a user with information for producing the proteins that are not mapped and the relations between the proteins that are not mapped.
